(19) United States
(12) Patent Application Publication
(10) Pub. No.: US 2017/0354721 A1
(43) Pub. Date: Dec. 14, 2017

(54) WOUND HEALING COMPOUND

(71) Applicant: Loubert S. Suddaby, Orchard Park, NY (US)

(72) Inventor: Loubert S. Suddaby, Orchard Park, NY (US)

(21) Appl. No.: 15/176,853

(22) Filed: Jun. 8, 2016

(51) Int. Cl. A61K 38/48; A61K 38/14; A61K 9/00; A61K 31/546; A61K 31/7036; A61K 31/7056

(52) U.S. Cl. CPC A61K 38/4833; A61K 31/7036; A61K 31/7056; A61K 9/0019; A61K 31/546; A61K 38/14; C12Y 304/21005

(57) ABSTRACT

The invention presented is an injectable wound healing compound that includes at least one antibiotic, a hemostatic agent, and an injectable flowable matrix. The antibiotic and the hemostatic agent are mixed in the injectable flowable matrix. The wound healing compound is injected onto an open wound using a syringe or syringe-like instrument. In one form the invention is a kit that includes an injecting instrument. Also disclosed is a method for making the wound healing compound.

### FIELD OF THE INVENTION

The invention relates to systems and methods of compounding one or more of various hemostatic agents with one or more clinically useful antibiotics for the express synergistic purpose of enhancing wound healing and preventing infection in both humans and animals.

### BACKGROUND OF THE INVENTION

Wound infections contribute markedly to extra days of hospitalization and related costs. The American Journal of Infection Control estimates that nearly one million additional inpatient days and $1.6 billion in extra costs occur as result of these serious operative complications. Post-operative wound infection occurs in about 2% of surgical procedures and accounts for 20% of health care associated infections. The estimated cost per infection alone ranges between $11,000 and $35,000.

Common predecessors of post-operative wound infection include hematomas and seromas which can cause the surgical incision to separate or dehisce thereby allowing bacteria to enter a wound and multiply in the retained fluid which serves as deal medium for pathogens to grow because of its warmth and wealth of nutrients. In addition, the increased use of low dose heparin therapy in surgical patients to prevent deep vein thrombosis post-operatively has further contributed to the development of post-operative hematomas.

Present measures employed to lessen the effect of hematoma/seroma formation involve the placement of drains or thrombotic agents in the surgical would. The problem here is that these very agents are in fact foreign bodies that can in turn serve as a nidus or breeding ground for infection. Additionally, once a hematoma or seroma forms in a wound, any prophylactic intravenous antibiotic given is much less likely to be effective because the pressure of the wound hematoma/seroma fluid connection on surrounding tissues inhibits the vascular distribution of the antibiotic to the wound, or because of timing of the antibiotic dosage insufficient antibiotic enters the wound to deter pathogens that are floating in seroma or hematoma fluid within a wound post-operatively, or that enter a wound later when spontaneous drainage of retained fluids in the wound occurs.

The physiology of hemostasis is well understood and many topical agents known in the art have been designed to facilitate the physiology of the hemostatic cascade. However, because the hemostatic agents have finite time in the wound to exert their biologic effect, they may also serve and have been implicated as forming a nidus for infection because they form a defacto foreign body when inserted into any wound.

From the foregoing, it is clear that hemostasis is important to achieving a good surgical outcome since the presence of fluid in a wound can both leak through the skin to provide a portal of entry and a medium of active bacteria, but also because of pressure exerted by hematoma on wound edges inhibits the ingress of white cells and prophylactic intravenously administered antibiotics needed to prevent infection. It is also clear that many present day hemostatic agents have evolved little since the early 1900's and while largely absorbable, form a nidus for infection during the critical portion of wound healing when infection most often occurs.

Therefore, it would be a significant improvement in the art to have a hemostatic agent capable of being place within a wound that would not only prevent hematoma and seroma formation, but would also carry along with it direct bactericidal or bacteriostatic properties which would disseminate with the wound in sufficient quantities to mitigate the deleterious effects of post-operative wound infections as well as to counteract the foreign body effect that hemostatic agents provide vis-a-vis these types of infections.

### SUMMARY OF THE INVENTION

The present invention broadly comprises an injectable wound healing compound including: at least one antibiotic; a hemostatic agent; and, an injectable flowable matrix. The antibiotic and said hemostatic agents are mixed in the injectable flowable matrix. In one embodiment, the injectable flowable matrix is a gelatin matrix. The hemostatic agent and the antibiotic agent each can be reconstituted using sterile water or sterile saline.

In one embodiment, the present invention is a kit that includes at least one antibiotic, a hemostatic agent, an injectable flowable matrix, and an injecting instrument such as a syringe or syringe-like instrument. In a preferred embodiment, the kit includes at least one container of sterile liquid such as water or saline for reconstitution or the antibiotic and/or the hemostatic agent.

The present invention also broadly comprises a method of making a wound healing compound having an antibiotic comprising: adding a hemostatic agent to a sterile liquid to form a hemostatic solution; mixing the hemostatic solution with a flowable matrix to form a hemostatic agent/flowable matrix mixture; reconstituting an antibiotic in sterile water; and, combining the reconstituted antibiotic in the hemostatic agent/flowable matrix to form the wound healing compound.

One object of the invention is to provide a compound that combines a hemostatic agent and at least one antibiotic in a compound deliverable to an open wound.

A second object of the invention is to reduce infection caused by application of a hemostatic agent to an open wound.

### DETAILED DESCRIPTION OF EMBODIMENTS OF THE INVENTION

At the outset, it should be appreciated that like drawing numbers on different drawing views identify identical structural elements of the invention. It also should be appreciated that figure proportions and angles are not always to scale in order to clearly portray the attributes of the present invention.

While the present invention is described with respect to what is presently considered to be the preferred embodiments, it is understood that the invention is not limited to the disclosed embodiments. The present invention is intended to cover various modifications and equivalent arrangements included within the spirit and scope of the appended claims.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. It should be appreciated that the term “substantially” is synonymous with terms such as “nearly”, “very nearly”, “about”, “approximately”, “around”, “bordering on”, “close to”, “essentially”, “in the neighborhood of”, “in the vicinity of”, etc., and such terms may be used interchangeably as appearing in the specification and claims. It should be appreciated that the term “proximate” is synonymous with terms such as “nearby”, “close”, “adjacent”, “neighboring”, “immediate”, “adjoining”, etc., and such terms may be used interchangeably as appearing in the specification and claims. Although any methods, devices or materials similar or equivalent to those described herein can be used in the practice or testing of the invention, the preferred methods, devices, and materials are now described.

The present invention is directed to an injectable wound healing compound containing at least one wound healing constituent, such as a hemostatic agent, and at least one antibiotic and/or antimicrobial component. In one preferred embodiment, the invention comprises an injectable flowable matrix (“matrix”) with the hemostatic agent and the antibiotic mixed in the matrix. By injectable is meant that the wound healing compound is delivered to its target, usually an open wound, by a syringe or syringe-like instrument. Such syringes are well known in the art.

Fibrin hemostatic sealants were first introduced in 1909 by Bergel and have continued to be refined to the present day. TISSEEL® (Baxter International Inc., Deerfield, Ill.) is perhaps the best known of this type of hemostatic agent.

Gelatin based hemostatic agents that include thrombin are perhaps more ubiquitous in the modern operating room. Present day iterations include FLOSEAL® (Baxter International, Inc.) and SURGIFLO® (Ethicon, Summerfield, N.J.) which is a mixture of thrombin reconstituted with water or saline with gelatin matrix granules allowing the hemostatic mixture to be administered as a flowable paste or gelfoam. The latter is administered as a sponge that can be cut into any shape to fit into a wound. However, both the paste and gelfoam can eventually act as a potential nidus for wound infection.

The exact chosen antibiotic(s) used in the wound healing compound depends on the sight of the surgical procedure being performed as certain areas of the body may be colonized by a specific organism that is more likely to invade a wound, or a certain surgical procedure may carry a statistically higher probability of being infected by a specific pathogen. For example, spinal surgery is most often complicated by gram positive organisms, such a Staphylococcus aureus or epidermidis infections, whereas abdominal surgery may be more often complicated by gram negative pathogens such as E. coli. Combinations of antibiotics or broad spectrum antibiotics could be used in cases where multiple types of pathogens might be found.

Gelatin based hemostatic agents are more ubiquitous in operating rooms and include FLOSEAL® (Fusion Medical Technologies, Inc., Mountain View, Calif.) and oxidized cellulose products such as SURGICEL® (Johnson & Johnson Corp., New Brunswick, N.J.).

The preferred embodiment would mate a particular antibiotic with a particular hemostatic agent for a specific operation. For example, vancomycin, bacitracin, erythromycin, mupirocin might be chosen to be used in operative procedures where gram positive organisms like s. aureus or epidermidis might be encountered. Likewise, gentamycin or polymixin B might be chosen form in abdominal procedures, for example, where gram negative organisms are more likely to be encountered.

Additionally, for plastic surgical procedures confined to skin, silver sulfadizone impregnated in a hemostatic agent may be chosen because of its superior topical effect.

The type of hemostatic agent chosen might also be varied to fit the surgery being performed. Endoscopic procedures would more likely employ agents with flowable properties such as fibrin sealant or paste like gelatin matrix combinations because they can be squeezed or injected along the portals used in this type of minimally invasive surgery.

Open wounds lend themselves to be secured with woven oxidized cellulose or layers of gelatin sponge applied directly to the wound surfaces. More superficial wounds such as those seen in plastic surgery may employ powder agents like microfibrillar collagen. In each case, the chosen hemostatic agent would be mated with an appropriate topical antibiotic suitable to counteract the most probable infectious organisms to be encountered with multiple or at least broad spectrum antibiotics chosen to treat cases where either unknown organisms or multiple different types of organisms might be encountered. For example, fluoroquinolones which have spectrum of activity which can include both gram positive and gram negative organisms.

The antibiotics, in turn, will be impregnated onto or with the hemostatic agents at an amount sufficient to provide at least a minimally inhibitive concentration for several days or until the hemostatic agent has been fully absorbed. The antibiotic can either be premixed with the hemostatic agent or mixed in the operating room under sterile conditions once the test combination of agent and antibiotic has been selected.

In one embodiment, the wound healing compound (“compound”) comprises an antibiotic, a hemostatic agent, and, an injectable flowable matrix (“matrix”) with the antibiotic and the hemostatic agent mixed in the injectable flowable matrix. In one embodiment of the compound, the injectable flowable matrix can be a gelatin matrix. In an alternate embodiment, the matrix can be an oxidized cellulose matrix.

In a preferred embodiment, the wound healing compound would include 2-4 ml of sterile water or sterile saline to which is added 20,000-40,000 IU's (International Units) of thrombin which acts as a hemostatic agent. This thrombin solution is mixed with a flowable gelatin matrix or a flowable oxidized cellulose matrix measuring in the amount of 7-14 ml.

Preferably, the entire thrombin/flowable agent mixture (“the mixture”) is placed in a 10 ml, 15 ml, or 20 ml syringe for a single use, depending on the size of the wound. People of ordinary skill in the art will recognize that syringes of other sizes can also be used, again depending on the size of the wound. In addition, it will be recognized that more than one syringe may be used for any one wound.

An antibiotic is added to the thrombin-flowable agent mixture. Examples of suitable antibiotics include, but are not limited to, are vancomycin, ancef, clindamycin, gentamycin and combinations of these antibiotics.

Preferably, the antibiotic is added to the thrombin-flowable agent mixture in an operating room or other sterile setting. However, the wound healing mixture can be applied in nonsterile settings such as accidents, combat situations, etc. when required.

### EXAMPLE

In an operating room setting, the vancomycin is reconstituted at room temperature (˜25° C.). Vancomycin is most effective at or slightly exceeding a concentration of 16 mcg/ml of water or saline if all ranges of bacterial infection are to be inhibited. To insure the Minimal Inhibitory Concentration (MIC) for vancomycin is exceeded, the total amount of vancomycin added to the mixture ranges from 1 mg to 5000 mg with the preferred amount being 500 mg for a 10 ml syringe, 750 mg for a 15 ml syringe, and 1000 for a 20 ml syringe. People having skill in the art will recognize that the concentrations of vancomycin can be adjusted for different sized syringes. The reconstituted vancomycin is added to the thrombin-flowable agent mixture to form a wound healing compound (“compound”). The compound is applied directly to the target wound. The Minimum Inhibitory Concentration (MIC) is defined as the lowest concentration of antimicrobial that will inhibit the visible growth of a microorganism after an overnight incubation. Related to this is the minimum bactericidal concentration (MBC), which is the lowest concentration of antimicrobial agent that will prevent the growth of an organism after subculture on to antibiotic free media.

Depending on the infection agent, other antibiotics may be added to the mixture to form the wound healing compound. In the lab, ancef sensitive organisms are inhibited at an MIC ranging from 1-4 mcg/ml. However, ancef is bactericidal a four times the MIC. Therefore, the preferred concentration (the MBC) would be at least 16 mcg/ml. The amount of ancef to be added to the same or similar thrombin-flowable agent mixture would range from 1 mg to 5000 mg to form the wound healing compound depending on the size of the wound to be treated. Within a similar range (1 mg-5000 mg) clindamycin may be added to the mixture with slightly higher concentrations because of the need for a higher MIC because certain staphylococcus organisms being relatively resistant to lower doses of clindamycin.

Persons of skill in the art will recognize that other antibiotics or combinations of antibiotics may be used to form the wound healing compound.

In one embodiment, the invention may be a kit that includes at least one antibiotic, a hemostatic agent, an injectable flowable matrix and, an injecting instrument such as a syringe. In a preferred embodiment, the kit will also include sterile liquid for reconstituting the hemostatic agent. In a more preferred embodiment, the kit may also include an additional sterile liquid for reconstituting the antibiotic(s).

Thus it is seen that the objects of the invention are efficiently obtained, although changes and modifications to the invention should be readily apparent to those having ordinary skill in the art, which changes would not depart from the spirit and scope of the invention as claimed.

1. An injectable wound healing compound comprising:
at least one antibiotic;
a hemostatic agent; and,
an injectable flowable matrix;
wherein said antibiotic and said hemostatic agent are mixed in said injectable flowable matrix.

2. The injectable wound healing compound as recited in claim 1 wherein said flowable matrix is a gelatin matrix.

3. The injectable wound healing compound as recited in claim 1 wherein said flowable matrix is an oxidized cellulose matrix.

4. The injectable wound healing compound as recited in claim 1 further comprising sterile water wherein said hemostatic agent is added to said sterile water prior to said mixing in said flowable matrix.

5. The injectable wound healing compound as recited in claim 1 wherein said hemostatic agent is thrombin.

6. The injectable wound healing compound as recited in claim 1 wherein said antibiotic is vancomycin.

7. The injectable wound healing compound as recited in claim 1 wherein said antibiotic is ancef.

8. The injectable wound healing compound as recited in claim 1 wherein said antibiotic is clindamycin.

9. The injectable wound healing compound as recited in claim 1 wherein said antibiotic is gentamycin.

10. The injectable wound healing compound as recited in claim 1 wherein said at least one antibiotic is a combination of one or more of vancomycin, ancef, clindamycin, or gentamycin.

11. A kit for treating wounds comprising:
at least one antibiotic;
a hemostatic agent;
an injectable flowable matrix and,
an injecting instrument.

12. The kit for treating wounds as recited in claim 11 wherein said injecting instrument is a syringe.

13. The kit for treating wounds as recited in claim 11 further comprising at least one sterile liquid.

14. The kit for treating wounds as recited in claim 11 wherein said hemostatic agent is thrombin.

15. The kit for treating wounds as recited in claim 11 wherein said at least one antibiotic is selected from the group consisting of vancomycin, ancef, clindamycin, gentamycin and combinations thereof.

16. A method of making a wound healing compound having an antibiotic comprising:
adding a hemostatic agent to a sterile liquid to form a hemostatic solution;
mixing said hemostatic solution with a flowable matrix to form a hemostatic agent/flowable matrix mixture;
reconstituting an antibiotic in sterile liquid; and,
combining said reconstituted antibiotic in said flowable matrix to form said wound healing compound.

17. The method of making a wound healing compound having an antibiotic as recited in claim 16 wherein said sterile liquid is sterile water.

18. The method of making a wound healing compound having an antibiotic as recited in claim 16 wherein said sterile liquid is sterile saline.

19. The method of making a wound healing compound having an antibiotic as recited in claim 16 wherein said antibiotic is elected from the group consisting of vancomycin, ancef, clindamycin, and gentamycin and combinations thereof.
